(19) United States
(12) Patent Application Publication
(10) Pub. No.: US 2018/0372640 A1
(43) Pub. Date: Dec. 27, 2018

(54) IMAGING DEVICE AND IMAGING METHOD

(71) Applicant: FUJIFILM Corporation, Tokyo (JP)

(72) Inventor: Yasuhisa KANEKO, Ashigarakami-gun (JP)

(73) Assignee: FUJIFILM Corporation, Tokyo (JP)

(21) Appl. No.: 16/119,492

(22) Filed: Aug. 31, 2018

Related U.S. Application Data
(63) Continuation of application No. PCT/JP2017/003695, filed on Feb. 2, 2017.

(30) Foreign Application Priority Data
Mar. 31, 2016 (JP) 2016-070143

(51) Int. Cl. G01N 21/64; G01N 33/48

(52) U.S. Cl. CPC G01N 21/6456; G01N 21/6428; G01N 2201/0627; G01N 2021/6441; G01N 2021/6471; G01N 33/48

(57) ABSTRACT

An imaging device includes a filter group including a first multi-bandpass filter, a dichroic mirror, and a second multi-bandpass filter, the first multi-bandpass filter transmitting excitation light rays having a plurality of wavelengths that excite a plurality of kinds of fluorescent dyes from among a light rays having a plurality of wavelengths emitted from a first light source, the dichroic mirror radiating excitation light rays having the plurality of wavelengths that have been transmitted through the first multi-bandpass filter toward a cell that has been stained with the plurality of kinds of fluorescent dyes emitting fluorescences of different wavelengths and transmitting the fluorescences of different wavelengths that are emitted from the cell due to the excitation light rays having the plurality of wavelengths, and the second multi-bandpass filter transmitting the fluorescences of different wavelengths that have been transmitted through the dichroic mirror.

### CROSS-REFERENCE TO RELATED APPLICATIONS

This application is a Continuation of PCT International Application No. PCT/JP2017/003695 filed on Feb. 2, 2017, which claims priority under 35 U.S.C § 119(a) to Japanese Patent Application No. 2016-070143 filed on Mar. 31, 2016. Each of the above application(s) is hereby expressly incorporated by reference, in its entirety, into the present application.

### BACKGROUND OF THE INVENTION

### 1. Field of the Invention

The present invention relates to an imaging device and an imaging method.

### 2. Description of the Related Art

In order to observe a cell having a plurality of fluorescent dyes bound thereto by immunostaining, a fluorescence microscope and the like are used as imaging devices.

It is described in JP2008-015059A that light from a laser light source is condensed by a microlens, light having an optional wavelength is transmitted by a dichroic mirror as excitation light, a sample is irradiated with the excitation light via a pinhole, and a fluorescence from the sample is imaged with a charge-coupled device (CCD) camera.

Furthermore, it is described in JP2009-058304A that a plurality of kinds of fluorescences emitted from a sample is transmitted through a wavelength-variable liquid crystal spectral filter that produces transmitted light having a variable wavelength range, and the fluorescences are imaged by a detector.

### SUMMARY OF THE INVENTION

Meanwhile, an imaging device that images a fluorescence emitted from a sample is required to image fluorescences of different wavelengths by one-time imaging, from the viewpoint of shortening the time.

However, JP2008-015059A does not disclose a specific configuration of an imaging element for imaging a fluorescence. Furthermore, in JP2009-058304A, imaging is achieved while the wavelength range of the light that has been transmitted through the wavelength-variable liquid crystal spectral filter is varied. Thus, JP2009-058304A discloses neither the imaging of fluorescences of different wavelengths by one-time imaging nor a specific configuration of an imaging element that images fluorescences of different wavelengths by one-time imaging.

The invention was achieved in view of such circumstances, and it is an object of the invention to provide an imaging device capable of imaging a plurality of fluorescences from a cell by one-time imaging, and an imaging method. It is another object of the invention to provide an imaging device capable of imaging, by one-time imaging, a plurality of fluorescences emitted from a cell and transmitted light that has been transmitted through the cell, and an imaging method.

According to an aspect of the invention, there is provided an imaging device comprising: at least one housing unit for holding and housing a cell that has been stained with a plurality of kinds of fluorescent dyes emitting fluorescences of different wavelengths on a flat holding surface; a first light source simultaneously emitting light rays having a plurality of wavelengths; a filter group including a first multi-bandpass filter, a dichroic mirror, and a second multi-bandpass filter, the first multi-bandpass filter selectively transmitting excitation light rays having a plurality of wavelengths that excite the plurality of kinds of fluorescent dyes from among the light rays having the plurality of wavelengths emitted from the first light source, the dichroic mirror radiating the excitation light rays having the plurality of wavelengths that have been transmitted through the first multi-bandpass filter toward the cell and transmitting the fluorescences of different wavelengths that are emitted from the cell due to the excitation light rays having the plurality of wavelengths, and the second multi-bandpass filter transmitting the fluorescences of different wavelengths that have been transmitted through the dichroic mirror; an objective lens condensing the excitation light rays having the plurality of wavelengths and amplifying the fluorescences of different wavelengths; and an imaging element having a plurality of sub-pixels in each pixel, and imaging the fluorescences of different wavelengths that have been transmitted through the second multi-bandpass filter.

It is preferable that the imaging element is a color imaging element.

It is preferable that the color imaging element is a single plate type imaging element having a red filter, a green filter, and a blue filter.

It is preferable that the first light source, the filter group, and the imaging element are disposed on the opposite side of the holding surface with respect to the housing unit.

It is preferable that the first multi-bandpass filter and the second multi-bandpass filter are each formed from a triple bandpass filter.

It is preferable that the first light source is composed of a plurality of light sources.

It is preferable that the imaging device further comprises a control unit for controlling the amounts of light from the plurality of light sources independently.

It is preferable that the first light source is a light source including at least a green light emitting diode, a blue light emitting diode, or a violet light emitting diode.

It is preferable that the housing unit is one of the housing units of a container having a plurality of housing units.

According to another aspect of the invention, there is provided an imaging device comprising: at least one housing unit for holding and housing a cell that has been stained with a plurality of kinds of fluorescent dyes emitting fluorescences of different wavelengths on a flat holding surface; a first light source simultaneously emitting light rays having a plurality of wavelengths; a second light source disposed on the opposite side of the first light source with respect to the housing unit and emitting transmitted light having a single wavelength; a filter group including a first multi-bandpass filter, a dichroic mirror, and a second multi-bandpass filter, the first multi-bandpass filter selectively transmitting excitation light rays having a plurality of wavelengths that excite the plurality of kinds of fluorescent dyes from among the light rays having the plurality of wavelengths emitted from the first light source, the dichroic mirror radiating the excitation light rays having the plurality of wavelengths that have been transmitted through the first multi-bandpass filter toward the cell and transmitting the plurality of fluorescences of different wavelengths that are emitted from the cell due to the excitation light rays having the plurality of wavelengths while also transmitting the transmitted light having the single wavelength emitted from the second light source, and the second multi-bandpass filter transmitting the fluorescences of different wavelengths that have been transmitted through the dichroic mirror and the transmitted light having the single wavelength; an objective lens condensing the excitation light rays having the plurality of wavelengths and amplifying the fluorescences of different wavelengths and the transmitted light; and an imaging element having a plurality of sub-pixels in each pixel, and imaging the fluorescences of different wavelengths that have been transmitted through the second multi-bandpass filter and the transmitted light having the single wavelength.

It is preferable that the first light source includes two light emitting diodes selected from a group consisting of a green light emitting diode, a blue light emitting diode, and a violet light emitting diode.

According to still another aspect of the invention, there is provided an imaging method, comprising: a step of housing a cell that has been stained with a plurality of kinds of fluorescent dyes emitting fluorescences of different wavelengths in at least one housing unit having a holding surface for holding the cell on a flat surface; a step of simultaneously emitting light rays having a plurality of wavelengths from a first light source; a step of selectively transmitting excitation light rays having a plurality of wavelengths that excite the plurality of kinds of fluorescent dyes from among the light rays having the plurality of wavelengths emitted from the first light source, through a first multi-bandpass filter, radiating the excitation light rays having the plurality of wavelengths that have been transmitted through the first multi-bandpass filter, toward the cell by a dichroic mirror, transmitting the fluorescences of different wavelengths emitted from the cell due to the excitation light rays having the plurality of wavelengths through the dichroic mirror, and transmitting the fluorescences of different wavelengths that have been transmitted through the dichroic mirror, through a second multi-bandpass filter; and a step of imaging the fluorescences of different wavelengths that have been transmitted through the second multi-bandpass filter, using an imaging element having a plurality of sub-pixels in each pixel.

According to still another aspect of the invention, there is provided an imaging method, comprising: a step of housing a cell that has been stained with a plurality of kinds of fluorescent dyes emitting fluorescences of different wavelengths in at least one housing unit having a holding surface for holding the cell on a flat surface; a step of simultaneously emitting light rays having a plurality of wavelengths from a first light source, and emitting transmitted light having a single wavelength from a second light source that is disposed on the opposite side of the first light source with respect to the housing unit; a step of selectively transmitting excitation light rays having a plurality of wavelengths that excite the plurality of kinds of fluorescent dyes from among the light rays having the plurality of wavelengths emitted from the first light source, through a first multi-bandpass filter, radiating the excitation light rays having the plurality of wavelengths that have been transmitted through the first multi-bandpass filter toward the cell by a dichroic mirror, transmitting the fluorescences of different wavelengths that have been emitted from the cell due to the excitation light rays having the plurality of wavelengths and the transmitted light having the single wavelength that is emitted from a second light source through the dichroic mirror, and transmitting the fluorescences of different wavelengths that have been transmitted through the dichroic mirror and the transmitted light having the single wavelength emitted from the second light source, through a second multi-bandpass filter; and a step of imaging the fluorescences of different wavelengths that have been transmitted through the second multi-bandpass filter and the transmitted light having the single wavelength emitted from the second light source, using an imaging element having a plurality of sub-pixels in each pixel.

According to the aspect of the invention, a plurality of fluorescences from a cell can be imaged by one-time imaging, and the time for imaging fluorescences emitted from a sample can be shortened.

According to the other aspect of the invention, a plurality of fluorescences from a cell and transmitted light that has been transmitted through the cell can be imaged by one-time imaging, and the time for imaging fluorescences emitted from a sample can be shortened.

### BRIEF DESCRIPTION OF THE DRAWINGS

FIG. 1 is a configuration diagram illustrating an imaging device of a first embodiment.

FIG. 2 is a partially magnified view of an imaging element.

FIG. 3 is a graph of spectra of light rays having a plurality of wavelengths that are simultaneously emitted from a first light source.

FIG. 4 is a graph of a spectroscopic spectrum of a first multi-bandpass filter.

FIG. 5 is a graph of the spectrum of excitation light.

FIG. 6 is a graph of the spectroscopic spectrum of a dichroic mirror.

FIG. 7 is a graph of spectra of fluorescences.

FIG. 8 is a graph of the spectroscopic spectrum of a second multi-bandpass filter.

FIG. 9 is a graph of the spectra of fluorescences.

FIG. 10 is a graph of the sensitivity characteristics of an imaging element having a filter.

FIG. 11 is a conceptual diagram of an image of fluorescences emitted from a cell, which have been obtained using an imaging element.

FIG. 12 is a configuration diagram of an imaging device of a second embodiment.

FIG. 13 is a graph of the spectra of light rays having a plurality of wavelengths that are simultaneously emitted from a first light source.

FIG. 14 is a graph of the spectroscopic spectrum of a first multi-bandpass filter.

FIG. 15 is a graph of the spectrum of excitation light.

FIG. 16 is a graph of the spectroscopic spectrum of a dichroic mirror.

FIG. 17 is a graph of the spectra of fluorescences.

FIG. 18 is a graph of the spectroscopic spectrum of a second multi-bandpass filter.

FIG. 19 is a graph of the spectra of fluorescences and transmitted light.

FIG. 20 is a conceptual diagram of an image of fluorescences from a cell and a phase difference image, which have been obtained using an imaging element.

### DESCRIPTION OF THE PREFERRED EMBODIMENTS

Hereinafter, preferred embodiments of the invention will be described in accordance with the attached drawings. The invention is explained by the following preferred embodiments. Modifications can be carried out by numerous techniques without deviating from the scope of the invention, and other embodiments in addition to those embodiments can be utilized. Therefore, all modifications made within the scope of the invention are included in the claims.

Here, in the diagrams, those parts designated by the same symbol are similar elements having similar functions. Furthermore, in the present specification, it should be noted that in a case in which a numerical range is represented using “to”, numerical values of the upper limit and the lower limit indicated with “to” are also included in the numerical range.

### First Embodiment

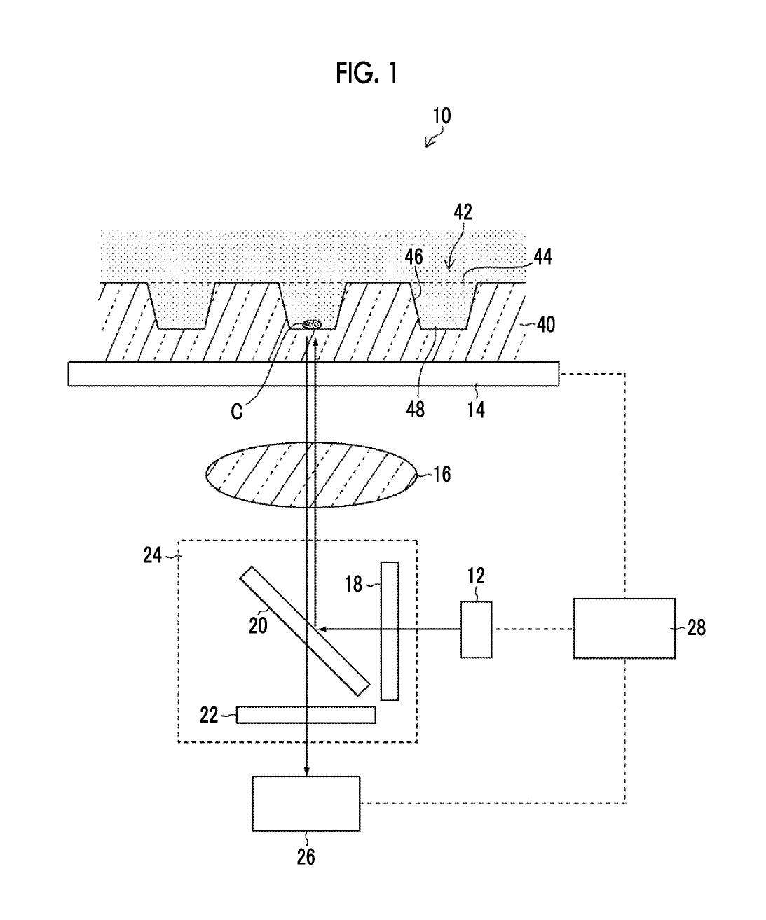

An imaging device and an imaging method of the first embodiment will be explained with reference to the drawings. FIG. 1 is a configuration diagram of an imaging device 10 of the present embodiment. The imaging device 10 illustrated in FIG. 1 is configured so as to be able to image fluorescences of different wavelengths that are emitted from a cell by one-time imaging.

The imaging device 10 comprises a first light source 12 for exciting fluorescent dyes bound to a cell C; a container 40 having a housing unit 42 for housing the cell C; a table 14 for placing the container 40; a filter group 24; an objective lens 16 disposed between the cell C and the filter group 24; and an imaging element 26 for imaging the fluorescence emitted from the cell C. The filter group 24 includes a first multi-bandpass filter 18, a dichroic mirror 20, and a second multi-bandpass filter 22. The housing unit 42 is formed on the surface of the container 40. In the example of FIG. 1, the container 40 has three housing units 42. However, the number of the housing units 42 is not limited to three, and the number may be two or less, or even four or more.

The control unit 28 controls the imaging performed by the imaging device 10. The control unit 28 is electrically connected to the table 14, the first light source 12, and the imaging element 26. The control unit 28 controls the operations of the table 14, the first light source 12, and the imaging element 26.

According to the present embodiment, the first light source 12, the filter group 24, and the imaging element 26 are disposed on the back surface side of the container 40. Therefore, the imaging device 10 can image the fluorescences of different wavelengths that are emitted from the cell C, from the back surface side of the container 40.

However, without being intended to be limited to this, it is also acceptable that the first light source 12, the filter group 24, and the imaging element 26 are disposed on the front surface side of the container 40. In this case, the imaging device 10 can image a plurality of fluorescences emitted from the cell C from the front surface side of the container 40.

The cell C imaged by the imaging device 10 is immunostained by an antigen-antibody reaction. An antigen-antibody reaction means that an antibody specifically binds to an antigen having a complementary structure. Immunostaining means that an antibody conjugated with a fluorescent dye is caused to bind to an antigen existing in a cell.

A fluorescent dye is excited by excitation light and emits a fluorescence. The fluorescence emitted due to the excitation light has a wavelength range on the longer wavelength side compared to the wavelength range of the excitation light.

According to the present embodiment, the cell C has at least a plurality of kinds of fluorescent dyes bound thereto by immunostaining. The plurality of kinds of fluorescent dyes is respectively excited by excitation light rays having different wavelength ranges and respectively emits fluorescences of different wavelengths.

Immunostaining includes a direct method and an indirect method. The direct method is a method by which a fluorescent dye is directly bound to an antibody and the antibody is caused to react with an antigen. Meanwhile, the indirect method is a method by which a fluorescent dye is not bound to an antibody that can specifically bind to the antigen to be detected (primary antibody), but the fluorescent dye is bound to an antibody that can specifically bind to the primary antibody (secondary antibody), and the fluorescence is detected.

As described above, the cell C is immunostained by an antigen-antibody reaction. For example, examples of an anti-human CD antibody include an anti-CD3 antibody, an anti-CD4 antibody, an anti-CD14 antibody, an anti-CD25 an antibody, and an anti-CD127 antibody. Examples of the fluorescent dye include 4′,6-diamidine-2′-phenylindole dihydrochloride (DAPI: 4′,6-diamidino-2-phenylindole), propidium iodide (PI: Propidiumlodide), Pyronin Y (Pyronin Y), fluorescein isothiocyanate (FITC: fluoresceinisothiocyanate), phycoerythrin (PE: phycoerythrin), allophycocyanin (APC: allophicocyanin), Texas Red (TR (registered trademark)), Hoechst 33342, 7-amino-actinomycin D (7-AAD), Cy3 (2′-Deoxycytidine 5′-triphosphoric acid), Cy5 (Sulfoindocyanine succinimidyl ester), DRAQ5 (registered trademark) (manufactured by Biostatus, Ltd.), Brilliant Violet 570, and Brilliant Violet 421.

The cell C is held in a state of being housed in at least one of the housing units 42 of the container 40 having a plurality of housing units 42. The housing unit 42 is formed by forming a recess in the container 40. The housing unit 42 has an opening 44, a lateral surface 46, and a flat holding surface 48. The flat holding surface 48 holds the cell C. The lateral surface 46 of the housing unit 42 has an inclined structure that spreads from the holding surface 48 toward the opening 44. By making the lateral surface 46 to have an inclined structure, the cell C can be easily housed inside the housing unit 42. It is desirable for the holding surface 48 that the site for holding the cell C is flat.

By making the holding surface 48 of the housing unit 42 for holding the cell C flat, it becomes easy to focus on the entirety of the cell at the time of imaging the cell C, and imaging of the cell C can be achieved reliably.

Regarding the material used for the container 40, a polymethacrylic acid ester such as polymethyl methacrylate; a polycarbonate; polystyrene; an Acrylonitrile-Butadiene-Styrene copolymerized rigid resin (ABS); aromatic polyesters such as polyethylene terephthalate and polybutylene terephthalate; various polyolefins such as polypropylene and a polycycloolefin; thermoplastic rigid resins such as a polysulfone, polyethersulfone, polyphenylene sulfide, polylactic acid, and a polyperfluoroalkoxy resin; thermosetting rigid resins such as polydimethylsiloxane; polytetrafluoroethylene; and the like can be used.

It is preferable that the imaging device 10 of the present embodiment includes a table 14 and a driving unit (not shown in the diagram), in order to have the container 40 moved (for example, in the X-direction, Y-direction, and Z-direction) to an optional position. The housing unit 42 housing the cell C of the container 40 can be moved to a position of observation by operating the table 14 and the driving unit. The driving unit is preferably an apparatus capable of moving the table 14 in the X-direction, Y-direction, and Z-direction.

The first light source 12 can simultaneously emit light rays having a plurality of wavelengths. In regard to the first light source 12, the structure, mode, and the like of the light source are not particularly limited as long as the first light source can simultaneously emit light rays having a plurality of wavelengths. The first light source 12 may be composed of a single light source, and light rays having a plurality of wavelengths may be emitted from the single light source. The first light source 12 may also be composed of a plurality of light sources emitting light rays having different wavelengths, so that light rays having a plurality of wavelengths are emitted from a plurality of light sources. Furthermore, the term “simultaneously” means that in a case in which the cell C is imaged, light rays having a plurality of wavelengths are included, and the light rays having a plurality of wavelengths may be emitted simultaneously or may be emitted separately.

The first light source 12 is not particularly limited; however, for example, a high-pressure mercury lamp, a high-pressure xenon lamp, a light emitting diode, a laser diode, a tungsten lamp, a halogen lamp, and a white light emitting diode can be used. Since the first light source 12 can simultaneously emit light rays having a plurality of wavelengths, the first light source 12 can selectively emit excitation light rays having a plurality of wavelengths that excite a plurality of kinds of fluorescent dyes in the cell C.

The filter group 24 includes a first multi-bandpass filter 18, a dichroic mirror 20, and a second multi-bandpass filter 22.

The first multi-bandpass filter 18 is disposed at a position facing the emission side of the first light source 12 so as to be inclined by about 90° with respect to the direction of advancement of light from the first light source 12. The first multi-bandpass filter 18 functions as an excitation filter. An excitation filter is an optical member that selectively transmits light having a wavelength that excites a fluorescent dye from among light rays having a plurality of wavelengths that are emitted from the first light source 12, and cuts off light rays having other wavelengths.

The first multi-bandpass filter 18 that constitutes the excitation filter is configured so as to selectively transmit only excitation light rays having a plurality of wavelengths (excitation light rays having at least two or more wavelengths) that excite the fluorescent dyes bound to the cell C, from among the light rays having a plurality of wavelengths that are emitted from the first light source 12. The first multi-bandpass filter 18 can be configured to include, for example, a glass substrate and a multilayer film of dielectric materials having different refractive indices.

The dichroic mirror 20 is disposed so as to be inclined by about 45° with respect to the direction of advancement of the excitation light rays having a plurality of wavelengths that have been transmitted through the first multi-bandpass filter 18. The dichroic mirror 20 is configured to reflect the excitation light rays having a plurality of wavelengths and radiates the reflected excitation light rays toward the cell C. Furthermore, the dichroic mirror 20 is configured to transmit fluorescences of different wavelengths that are emitted by a plurality of kinds of fluorescent dyes bound to the cell C, which have been excited by the excitation light rays having a plurality of wavelengths. That is, the dichroic mirror 20 is an optical member for separating excitation light from a fluorescence.

Since a fluorescence emitted due to excitation light has a wavelength range on the longer wavelength side compared to the wavelength range of the excitation light, it is made possible to transmit a fluorescence only by using the dichroic mirror 20. The dichroic mirror 20 can be configured to include, for example, a glass substrate and a multilayer film of dielectric materials having different refractive indices.

The second multi-bandpass filter 22 is disposed so as to be inclined by about 90° with respect to the direction of advancement of the fluorescences of different wavelengths that have been transmitted through the dichroic mirror 20. The second multi-bandpass filter 22 functions as a fluorescence filter. A fluorescence filter is an optical member that transmits only a fluorescence having a necessary wavelength range from the fluorescences of different wavelengths emitted from the cell C, and cuts off other light rays.

The second multi-bandpass filter 22 that constitutes the fluorescence filter can transmit only the fluorescences of different wavelengths emitted from the cell C without transmitting the excitation light. The second multi-bandpass filter 22 can be configured to include, for example, a glass substrate and a multilayer film of dielectric materials having different refractive indices.

The objective lens 16 is disposed between the filter group 24 and the cell C in order to condense the excitation light rays reflected at the dichroic mirror 20 and to amplify the fluorescences emitted from the cell C. Any lens that is used for optical measurement can be used as the objective lens 16.

The imaging element 26 images the fluorescences of different wavelengths that have been transmitted through the second multi-bandpass filter 22. The imaging element 26 is disposed so as to be inclined by about 90° with respect to the direction of advancement of the fluorescences of different wavelengths that have been transmitted through the dichroic mirror 20. The imaging element 26 is an electronic device that converts received light to electric signals.

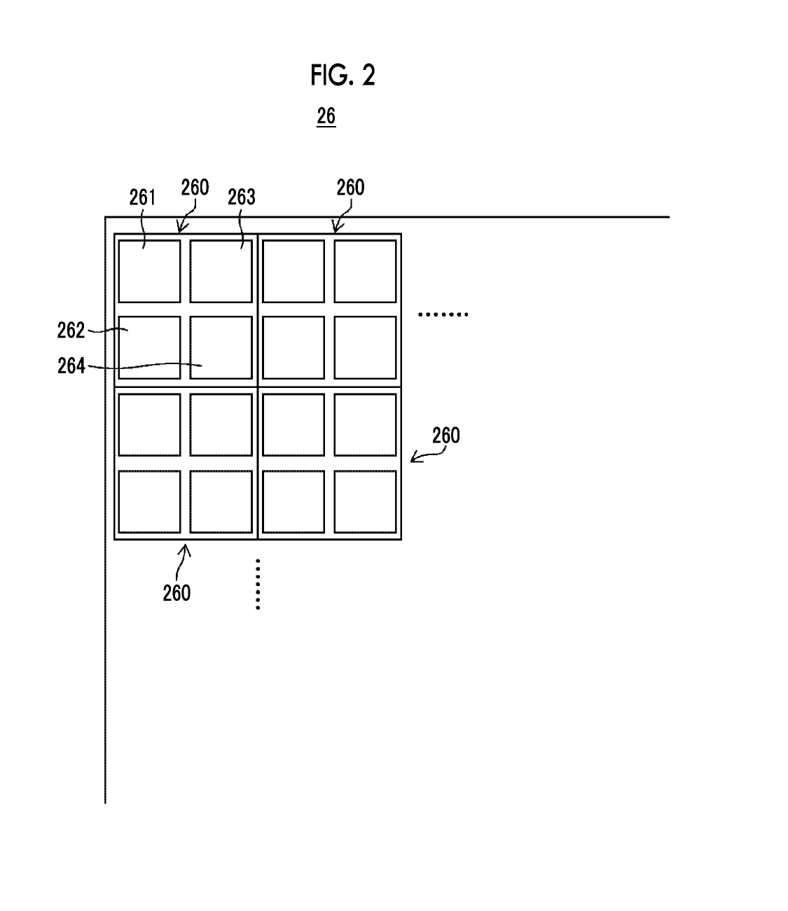

FIG. 2 is a partially magnified view of the imaging element 26. The imaging element 26 includes a plurality of pixels 260 as illustrated in FIG. 2. One pixel 260 has a plurality of sub-pixels 261, 262, 263, and 264. That is, a plurality of sub-pixels 261, 262, 263, and 264 constitutes one pixel 260.

The present embodiment illustrates the case in which the sizes of the sub-pixels 261, 262, 263, and 264 are the same; however, the sizes of the sub-pixels 261, 262, 263, and 264 can be modified as appropriate. Furthermore, the present embodiment exemplifies the case in which one pixel 260 has four sub-pixels 261, 262, 263, and 264; however, one pixel 260 may have three sub-pixels, or even five or more sub-pixels.

The intensities of the fluorescences obtained by a plurality of the sub-pixels 261, 262, 263, and 264 of the imaging element 26 are computed by the control unit 28, and the intensity of a fluorescence as one pixel 260 is calculated.

For example, in a case in which the imaging element 26 is caused to function as a single plate type color imaging element, a plurality of sub-pixels 261, 262, 263, and 264 can be incorporated into one pixel 260 by arranging a red filter, a green filter, and a blue filter above the one pixel 260. A red filter, a green filter, and a blue filter are optical filters that transmit only the respective colors. A single plate type color imaging element is a single imaging element provided with a red filter, a green filter, and a blue filter, with which a color image is obtained.

At the time of arranging the red filter, green filter, and blue filter, for example, it is preferable that a green filter is arranged on the sub-pixels 261 and 264, a blue filter is arranged on the sub-pixel 262, and a red filter is arranged on the sub-pixel 263. However, the arrangement is not limited to this and can be varied as appropriate.

An example of configuring the sub-pixels has been illustrated with an optical filter; however, the example is not limited to this. For example, the imaging element may be a phase-difference camera, in which four sub-pixels having diffraction gratings that are shifted from each other little by little constitute one pixel in order to image a phase difference image.

Next, in regard to an imaging method of using the imaging device 10 configured as explained above, the case in which the first light source 12 is composed of a green light emitting diode, a blue light emitting diode, and a violet light emitting diode will be described as an example.

First, a cell C is stained by immunostaining with a plurality of kinds of fluorescent dyes that emit fluorescences of different wavelengths. Next, in the step of housing, the cell C is housed on the holding surface 48 of at least one housing unit 42 of a container 40 having a plurality of housing units 42, each housing unit having a holding surface 48 on a flat surface.

Next, in the step of emitting, a green light emitting diode, a blue light emitting diode, and a violet light emitting diode, which constitute the first light source 12, are simultaneously caused to emit light. As a result, light rays having a plurality of wavelengths are simultaneously emitted from the first light source 12.

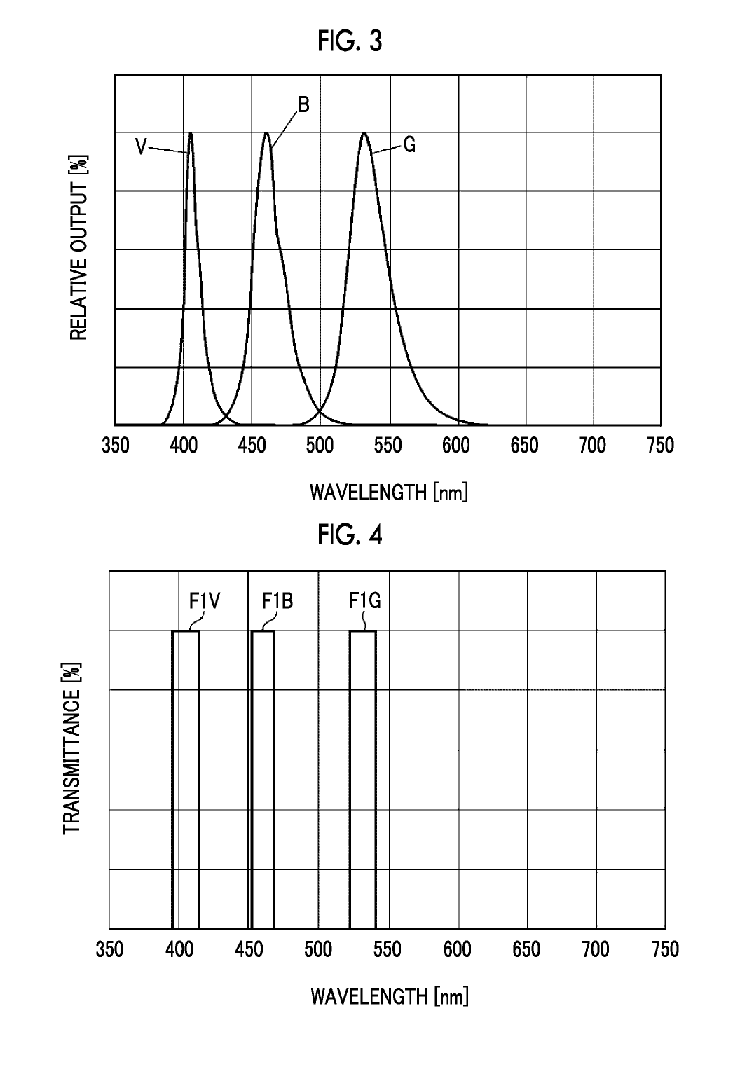

FIG. 3 is a graph of the spectra of light rays having a plurality of wavelengths that are simultaneously emitted from the first light source 12, and in the graph, the axis of ordinate represents the relative output (%) while the axis of abscissa represents the wavelength (nm). The green light emitting diode emits green light G having a peak wavelength in the range of from 490 nm to 560 nm. The blue light emitting diode emits blue light B having a peak wavelength in the range of from 430 nm to 490 nm. The violet light emitting diode emits violet light V having a peak wavelength in the range of from 380 nm to 430 nm.

The light rays having a plurality of wavelengths that are simultaneously emitted from the first light source 12 are not limited to the respective colored light rays emitted from the green light emitting diode, the blue light emitting diode, and the violet light emitting diode. The light rays having a plurality of wavelengths that are simultaneously emitted from the first light source 12 can be selected as appropriate according to the excitation light for the fluorescent dyes bound to the cell C.

In the present embodiment, the relative outputs of the green light G, the blue light B, and the violet light V are set to be constant; however, there are no limitations. For example, the control unit 28 controls the amounts of light from the green light emitting diode, the blue light emitting diode, and the violet light emitting diode independently. Thereby, the imaging device 10 can change the intensity of the fluorescence emitted from the cell C, which is an object of imaging, and can image the fluorescences emitted from the cell C more accurately.

Next, the excitation light rays having a plurality of wavelengths that excite the fluorescent dyes bound to the cell C from among the light rays having a plurality of wavelengths emitted from the first light source 12 are transmitted through a first multi-bandpass filter 18. FIG. 4 is a graph showing a spectroscopic spectrum of the first multi-bandpass filter 18, and in the graph, the axis of ordinate represents the transmittance (%) while the axis of abscissa represents the wavelength (nm). As shown in FIG. 4, the first multi-bandpass filter 18 has spectroscopic spectra of F1G, F1B, and F1V, which transmit only wavelength ranges that are narrower than the respective wavelength ranges of the green light G, blue light B, and violet light V, and the first multi-bandpass filter 18 can be configured as a triple bandpass filter.

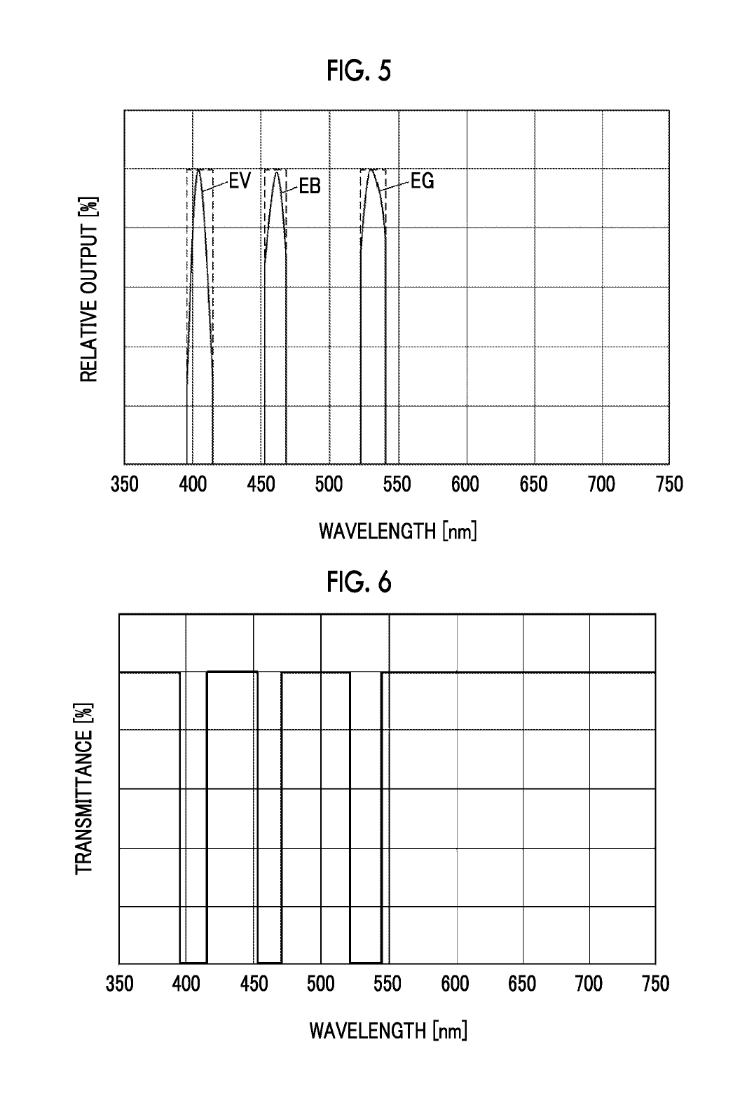

The first multi-bandpass filter 18 transmits excitation light rays of three wavelengths from among the light rays having a plurality of wavelengths emitted from the first light source 12. FIG. 5 is a graph of the spectra of three excitation light rays, and in the graph, the axis of ordinate represents the relative output (%) while the axis of abscissa represents the wavelength (nm). The first multi-bandpass filter 18 transmits an excitation light EG having a narrower range than the green light G that is radiated from the green light emitting diode. The first multi-bandpass filter 18 also transmits an excitation light EB having a narrower range than the blue light B that is radiated from the blue light emitting diode. The first multi-bandpass filter 18 also transmits an excitation light EV having a narrower range than the violet light V that is radiated from the violet light emitting diode.

FIG. 6 is a graph showing a spectroscopic spectrum of the dichroic mirror 20, and in the graph, the axis of ordinate represents the transmittance (%) while the axis of abscissa represents the wavelength (nm). As shown in FIG. 6, according to the spectroscopic characteristics of the dichroic mirror 20, the transmittances in the wavelength ranges corresponding to the excitation light rays EG, EB, and EV are set at low levels. Therefore, a plurality of excitation light rays EG, EB, and EV is reflected by the dichroic mirror 20 and is radiated toward the cell C.

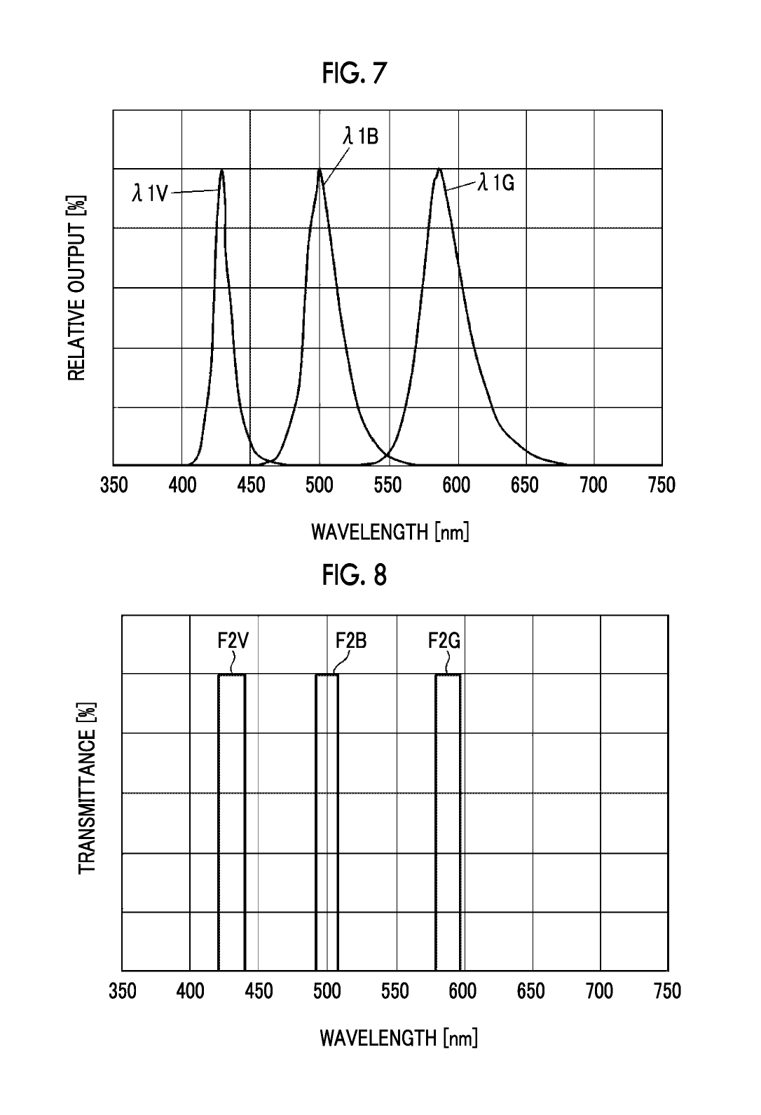

As a plurality of excitation light rays EG, EB, and EV excites a plurality of kinds of fluorescent dyes that are bound to the cell C, the plurality of kinds of fluorescent dyes bound to the cell C emits fluorescences of different wavelengths. FIG. 7 is a graph of the spectra of three fluorescences λ1G, λ1B, and λ1V that are emitted from a plurality of kinds of fluorescent dyes that has been excited by three excitation light rays EG, EB, and EV, and in the graph, the axis of ordinate represents the relative output (%) while the axis of abscissa represents the wavelength (nm). As shown in FIG. 7, the fluorescences λ1G, λ1B, and λ1V respectively have wavelength ranges on the longer wavelength side than the excitation light rays EG, EB, and EV.

The fluorescences λ1G, λ1B, and λ1V are transmitted through the objective lens 16 and the dichroic mirror 20 having the spectroscopic characteristics shown in FIG. 6 and reach a second multi-bandpass filter 22. Here, as shown in FIG. 6, according to the spectroscopic characteristics of the dichroic mirror 20, the transmittances in the wavelength ranges of the fluorescences λ1G, λ1B, and λ1V are high. Accordingly, the fluorescences λ1G, λ1B, and λ1V are transmitted through the dichroic mirror 20. FIG. 8 is a graph showing a spectroscopic spectrum of the second multi-bandpass filter 22, and in the graph, the axis of ordinate represents the transmittance (%) while the axis of abscissa represents the wavelength (nm). As shown in FIG. 8, the second multi-bandpass filter 22 has spectroscopic spectra of F2G, F2B, and F2V, which transmits light rays in narrower wavelength ranges than the wavelength ranges of the fluorescences λ1G, λ1B, and λ1V, and the second multi-bandpass filter 22 can be configured as a triple bandpass filter.

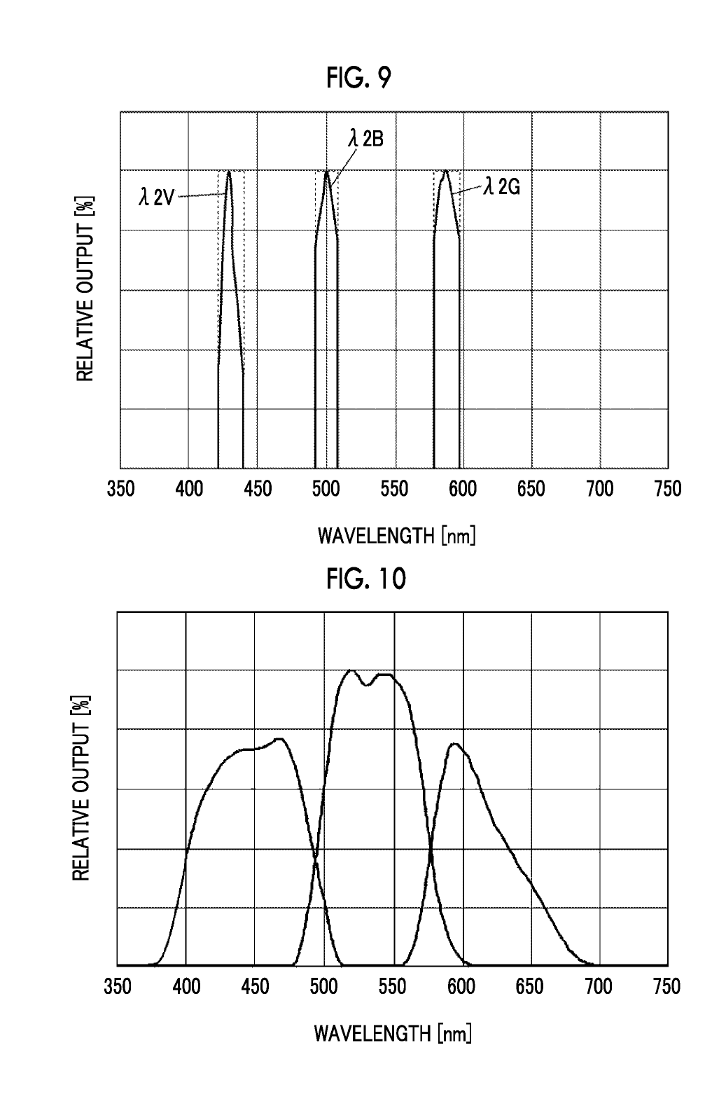

The fluorescence λ1G that has been emitted from the cell C and transmitted through the dichroic mirror 20 is treated such that light rays in unnecessary wavelength ranges are cut off upon being transmitted through the second multi-bandpass filter 22. The fluorescence λ1B that has been emitted from the cell C and transmitted through the dichroic mirror 20 is treated such that light rays in unnecessary wavelength ranges are cut off upon being transmitted through the second multi-bandpass filter 22. The fluorescence λ1V that has been emitted from the cell C and transmitted through the dichroic mirror 20 is treated such that light rays in unnecessary wavelength ranges are cut off upon being transmitted through the second multi-bandpass filter 22. FIG. 9 is a graph of the spectra of fluorescences λ2G, λ2B, and λ2V that have been transmitted through the second multi-bandpass filter 22, and in the graph, the axis of ordinate represents the relative output (%) while the axis of abscissa represents the wavelength (nm). Regarding the fluorescence λ1G, unnecessary wavelength ranges are cut off by the second multi-bandpass filter 22, and the fluorescence λ2G having a narrower wavelength range than the fluorescence λ1G is outputted from the second multi-bandpass filter 22. Regarding the fluorescence λ1B, unnecessary wavelength ranges are cut off by the second multi-bandpass filter 22, and the fluorescence λ2B having a narrower wavelength range than the fluorescence λ1B is outputted from the second multi-bandpass filter 22. Regarding the fluorescence λ1V, unnecessary wavelength ranges are cut off by the second multi-bandpass filter 22, and the fluorescence λ2V having a narrower wavelength range than the fluorescence λ1V is outputted from the second multi-bandpass filter 22.

Next, in the step of imaging, the fluorescences λ2G, λ2B, and λ2V that have been transmitted through the second multi-bandpass filter 22 are imaged by an imaging element 26 having a plurality of sub-pixels in each pixel. In the present embodiment, regarding the imaging element 26, a single plate type color imaging element in which a red filter, a green filter, and a blue filter constitute sub-pixels can be used. FIG. 10 is a graph showing the sensitivity characteristics of the imaging element 26 having a red filter, a green filter, and a blue filter that constitute sub-pixels, and in the graph, the axis of ordinate represents the relative output (%) while the axis of abscissa represents the wavelength (nm). For example, the red filter transmits light in the wavelength range of from 555 nm to 700 nm, the green filter transmits light in the wavelength range of from 470 nm to 605 nm, and the blue filter transmits light in the wavelength range of from 375 nm to 510 nm. However, the wavelength ranges that are transmitted by the red filter, the green filter, and the blue filter can be selected as appropriate.

The intensities of the fluorescences imaged by the imaging element 26 are inputted and stored in, for example, a control unit 28. The intensities of the fluorescences are computed by the control unit 28, and thus a color image is obtained.

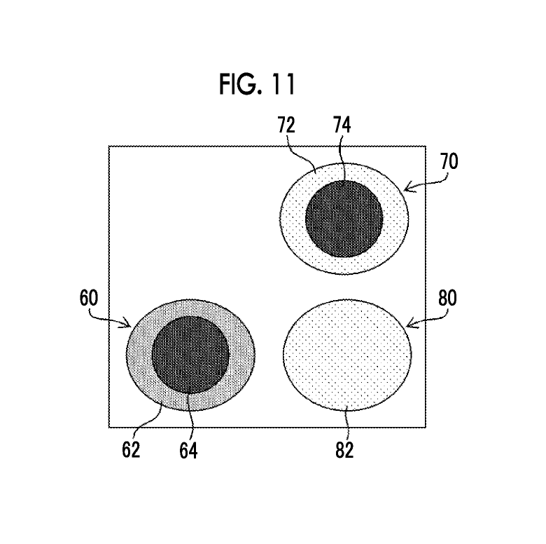

FIG. 11 is a conceptual diagram of an image of fluorescences emitted from a cell C, the image being obtained by the control unit 28 based on the intensities of fluorescences obtained by the imaging element 26. For example, a white blood cell 60, a nucleated red blood cell 70, and a red blood cell 80 are shown. In the white blood cell 60, the surface 62 emits blue light due to a fluorescent dye, while the internal nucleus 64 emits red light due to a fluorescent dye. In the nucleated red blood cell 70, the surface 72 emits green light due to a fluorescent dye, while the internal nucleus 74 emits red light due to a fluorescent dye. In the red blood cell (immature) 80, the surface 82 emits green light due to a fluorescent dye. Meanwhile, since this red blood cell does not have an internal nucleus, the red blood cell 80 does not emit red light.

In the present embodiment, since fluorescences of different wavelengths emitted from a cell C can be simultaneously imaged by one-time imaging as described above, the time for imaging the cell C can be shortened.

### Second Embodiment

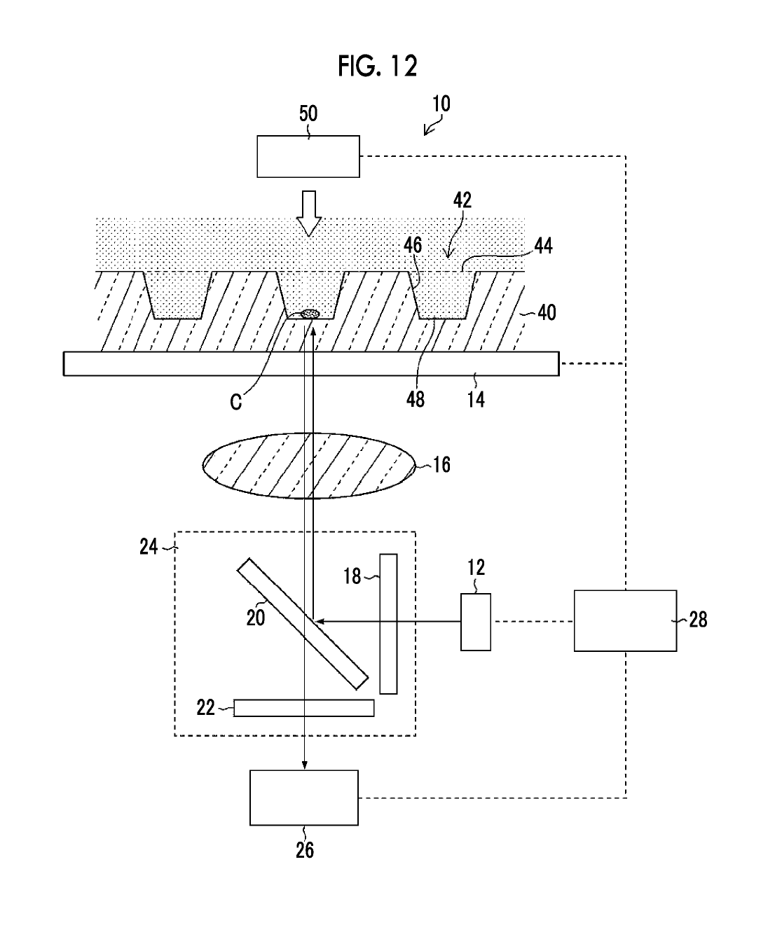

An imaging device of the second embodiment as illustrated in FIG. 12 and an imaging method will be described with reference to the drawings. The imaging device 10 is configured so as to be able to image a plurality of fluorescences and a bright field image from a cell by one-time imaging. Furthermore, configurations similar to the configurations of the first embodiment will be assigned with the same symbols, and further explanation may not be repeated.

The imaging device 10 comprises a first light source 12 for exciting the fluorescent dyes bound to a cell C; a container 40 having a housing unit 42 for housing the cell C; a table 14 for placing the container 40; a filter group 24; an objective lens 16 disposed between the cell C and the filter group 24; a second light source 50 for irradiating the cell C with transmitted light that has been transmitted through the cell C; and an imaging element 26 for imaging the fluorescences emitted from the cell C and the transmitted light that has been transmitted through the cell C. According to the present embodiment, similarly to the first embodiment, the filter group 24 includes a first multi-bandpass filter 18, a dichroic mirror 20, and a second multi-bandpass filter 22. Furthermore, the housing unit 42 is formed on the surface of the container 40. In the example of FIG. 12, the container 40 has three housing units 42. However, the number of the housing units 42 is not limited to three, and the number may be two or less, or even four or more. The second light source 50 is disposed on the front surface side of the container 40, and the first light source 12 is disposed on the back surface side of the container 40. That is, the second light source 50 is disposed on the opposite side of the first light source with respect to the housing units 42 formed in the container 40.

A control unit 28 controls the imaging performed by the imaging device 10. The control unit 28 is electrically connected to the table 14, the first light source 12, the imaging element 26, and the second light source 50. The control unit 28 controls the operations of the table 14, the first light source 12, the imaging element 26, and the second light source 50.

According to the present embodiment, fluorescences of different wavelengths can be imaged from the back surface side of the container 40, similarly to the first embodiment. Furthermore, a bright field image can also be imaged from the back surface side of the container 40. However, without being limited to this, it is also possible to image the fluorescences of different wavelengths emitted from the cell C and a bright field image of the cell C from the front surface side of the container 40.

Similarly to the first embodiment, the first light source 12 can simultaneously emit light rays having a plurality of wavelengths. Regarding the first light source 12, the structure, mode and the like of the light source are not particularly limited as long as the first light source 12 can simultaneously emit light rays having a plurality of wavelengths.

It is preferable that the first light source 12 includes two light emitting diodes selected from the group consisting of a green light emitting diode, a blue light emitting diode, and a violet light emitting diode. By having light rays having different wavelengths emitted from two light emitting diodes, fluorescences of two different wavelengths can be emitted from the fluorescent dyes bound to the cell C.

Regarding the second light source 50, the structure, mode and the like of the light source are not particularly limited as long as the second light source 50 can emit light having a wavelength different from those of the excitation light rays that excite the fluorescent dyes of the cell C and the fluorescences emitted from the fluorescent dyes. The second light source 50 is not particularly limited; however, for example, a high-pressure mercury lamp, a high-pressure xenon lamp, a light emitting diode, a laser diode, a tungsten lamp, a halogen lamp, and a white light emitting diode can be used. In a case in which the wavelength range of the light emitted from the second light source 50 includes the same wavelengths as those of the excitation light rays that excite the fluorescent dyes in the cell C or the fluorescences emitted from the fluorescent dyes, light rays having wavelengths that are different from those of the excitation light rays that excite the fluorescent dyes of the cell C and the fluorescences emitted from the fluorescent dyes can be radiated to the cell C by providing a bandpass filter (not shown in the diagram) between the cell C and the second light source.

Similarly to the first embodiment, the filter group 24 includes a first multi-bandpass filter 18, a dichroic mirror 20, and a second multi-bandpass filter 22.

The first multi-bandpass filter 18 functions as an excitation filter, which is an optical member; the dichroic mirror 20 functions as an optical member for separating excitation light from a fluorescence; and the second multi-bandpass filter 22 functions as a fluorescence filter, which is an optical member.

Furthermore, in the second embodiment, the dichroic mirror 20, and the second multi-bandpass filter 22 transmit the transmitted light that has been radiated from the second light source 50 and transmitted through the cell C. The transmitted light from the second light source 50, which has been transmitted through the cell C, constitutes a bright field image. Furthermore, the bright field image can also be obtained as a phase difference image by disposing a condenser for phase difference (doughnut-shaped slit) immediately before the second light source 50, which is a light source for transmitted light, and changing the objective lens to a lens for phase difference observation obtained by adding a phase ring to an objective lens.

The objective lens 16 is disposed between the filter group 24 and the cell C in order to condense the excitation light radiated from the dichroic mirror 20 and to amplify the fluorescences emitted from the cell C and the transmitted light. Regarding the objective lens 16, any lens that is used for optical measurement can be used.

The imaging element 26 images the fluorescences of different wavelengths that have been transmitted through the second multi-bandpass filter 22 and transmitted light having a single wavelength. The imaging element 26 can be configured similarly to the first embodiment, and as shown in FIG. 2, the imaging element 26 includes a plurality of pixels 260. One pixel 260 has a plurality of sub-pixels 261, 262, 263, and 264. That is, a plurality of sub-pixels 261, 262, 263, and 264 constitutes one pixel 260. In the present embodiment, similarly to the first embodiment, the sizes of the sub-pixels 261, 262, 263, and 264 may be modified as appropriate. One pixel 260 may have three sub-pixels, or even five or more sub-pixels.

Next, in regard to an imaging method of using the imaging device 10 configured as explained above, the case in which the first light source 12 is a light source including a blue light emitting diode and a violet light emitting diode, and the second light source 50 is a red light emitting diode, will be explained as an example. First, the cell C is stained with fluorescent dyes and is held on the holding surface 48.

In the step of emitting, the blue light emitting diode and the violet light emitting diode that constitute the first light source 12 are simultaneously caused to emit light, and thereby light rays having a plurality of wavelengths are simultaneously emitted from the first light source 12.

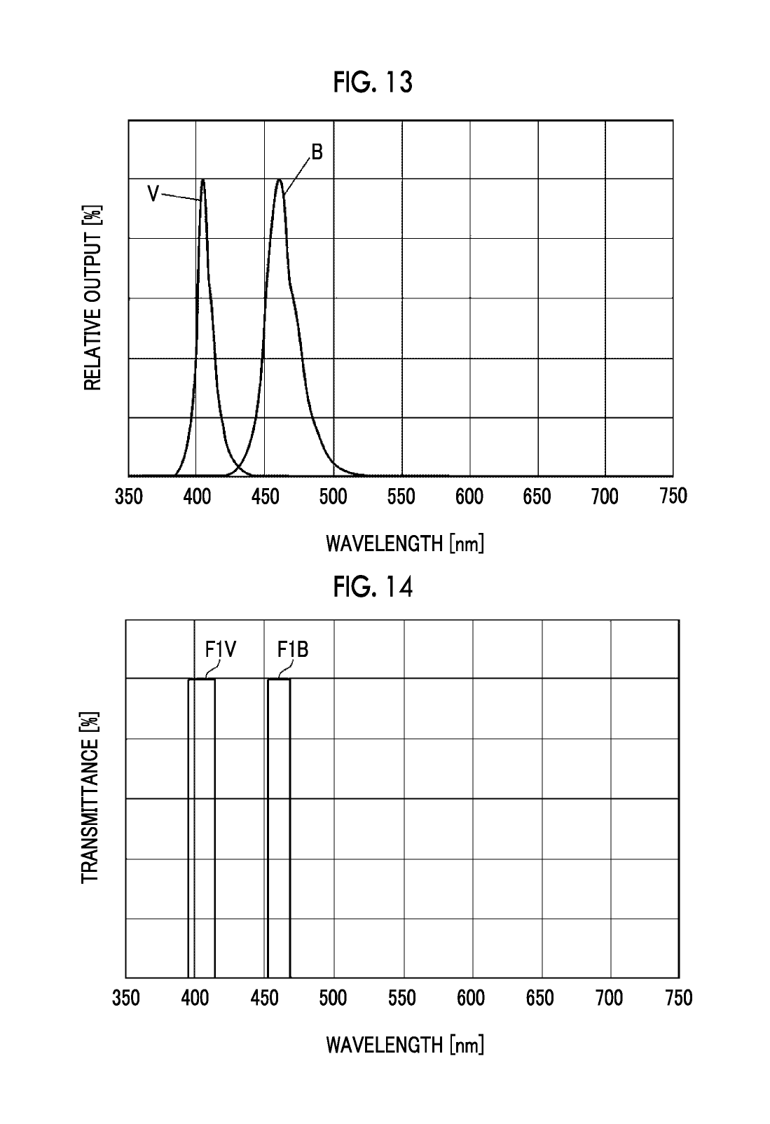

FIG. 13 is a graph of the spectra of light rays having a plurality of wavelengths that are simultaneously emitted from the first light source 12, and in the graph, the axis of ordinate represents the relative output (%) while the axis of abscissa represents the wavelength (nm). The blue light emitting diode emits blue light B having a peak wavelength of 470 nm. The violet light emitting diode emits violet light V having a peak wavelength of 405 nm.

The light rays having a plurality of wavelengths that are simultaneously emitted from the first light source 12 are not limited to the various colored light rays emitted from the blue light emitting diode and the violet light emitting diode. The light rays can be selected as appropriate according to the excitation light rays for the fluorescent dyes that are bound to the cell C.

Similarly to the first embodiment, the control unit 28 can control the amounts of light from the blue light emitting diode and the violet light emitting diode independently.

Next, the excitation light rays having a plurality of wavelength ranges, which excite the fluorescent dyes bound to the cell C, from among the light rays having a plurality of wavelengths emitted from the first light source 12 transmit the first multi-bandpass filter 18. FIG. 14 is a graph showing a spectroscopic spectrum of the first multi-bandpass filter 18, and in the graph, the axis of ordinate represents the transmittance (%) while the axis of abscissa represents the wavelength (nm). As shown in FIG. 14, the first multi-bandpass filter 18 has spectroscopic spectra of F1B and F1V, which transmits only wavelength ranges that are narrower than the respective wavelength ranges of the blue light B and the violet light V, and the first multi-bandpass filter 18 can be configured as a dual bandpass filter. In a case in which the spectrum does not overlap with the spectrum of transmitted light that will be described below, the first multi-bandpass filter 18 may also have a spectroscopic spectrum of green light F1G as shown in FIG. 4.

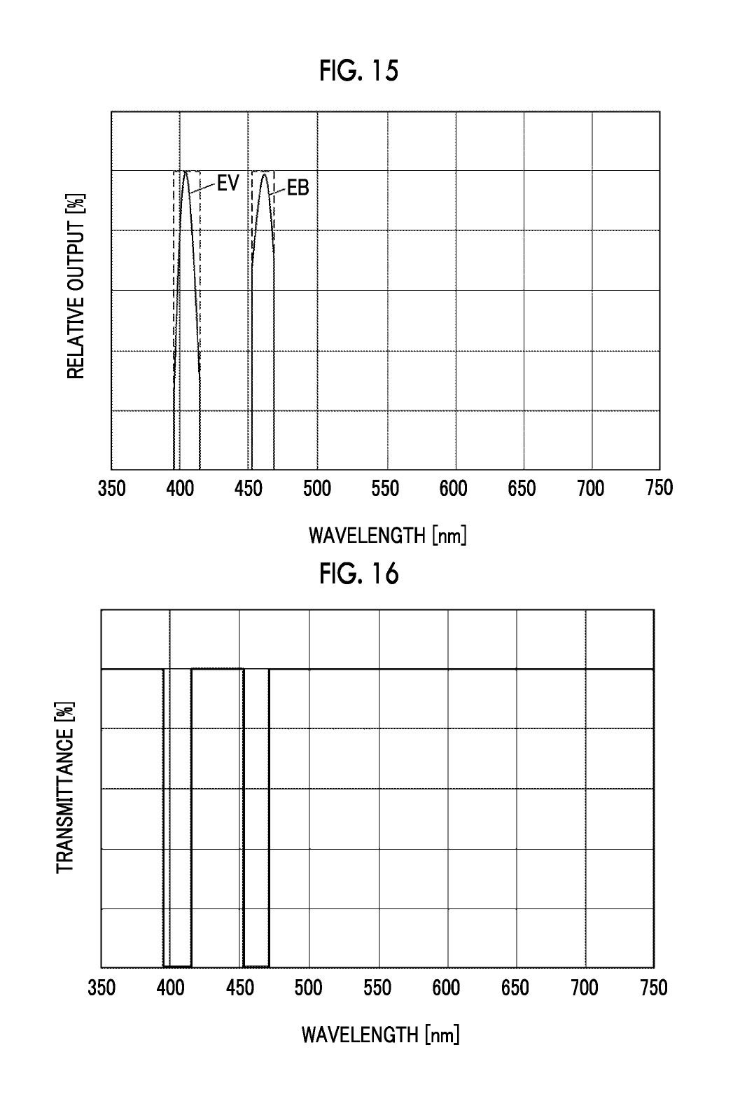

The first multi-bandpass filter 18 transmits excitation light rays having two wavelengths from among the light rays having a plurality of wavelengths emitted from the first light source 12. FIG. 15 is a graph of the spectra of two excitation light rays, and in the graph, the axis of ordinate represents the relative output (%) while the axis of abscissa represents the wavelength (nm). The first multi-bandpass filter 18 transmits excitation light EB having a narrower range than the blue light B that is radiated from the blue light emitting diode. Furthermore, the first multi-bandpass filter 18 transmits excitation light EV having a narrower range than the violet light V that is radiated from the violet light emitting diode.

FIG. 16 is a graph showing a spectroscopic spectrum of the dichroic mirror 20, and in the graph, the axis of ordinate represents the transmittance (%) while the axis of abscissa represents the wavelength (nm). As shown in FIG. 16, according to the spectroscopic characteristics of the dichroic mirror 20, the transmittances in the wavelength ranges corresponding to the excitation light rays EB and EV are set at low levels. Therefore, a plurality of excitation light rays EB and EV is reflected by the dichroic mirror 20 and is radiated toward the cell C. Meanwhile, in a case in which the spectrum does not overlap with the spectrum of transmitted light that will be described below, there may be a region where transmission does not occur at 520 nm to 550 nm, as shown in FIG. 6.

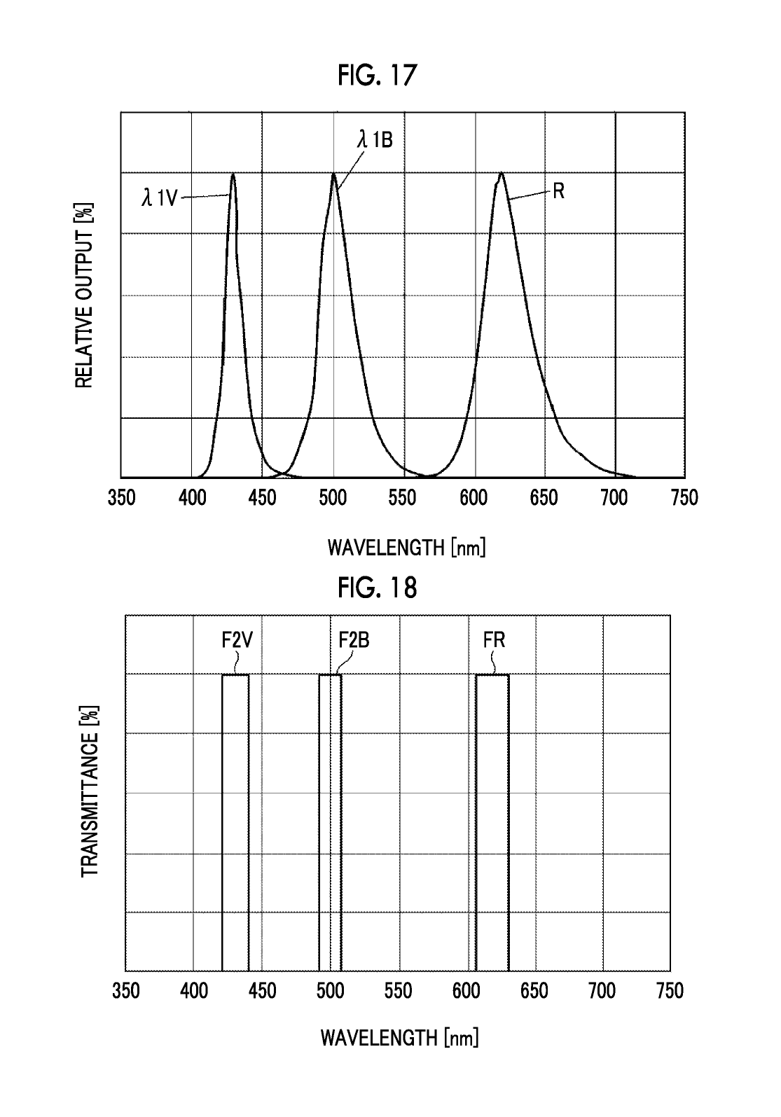

As a plurality of excitation light rays EB and EV excites a plurality of kinds of fluorescent dyes bound to the cell C, the plurality of kinds of fluorescent dyes bound to the cell C emits fluorescences of different wavelengths. FIG. 17 is a graph of the spectra of the fluorescences λ1B and λ1V emitted from a plurality of kinds of fluorescent dyes that has been excited by two excitation light rays EB and EV, and the transmitted light R from the second light source 50, and in the graph, the axis of ordinate represents the relative output (%) while the axis of abscissa represents the wavelength (nm). As shown in FIG. 17, the fluorescences λ1B and λ1V respectively have wavelength ranges on the longer wavelength side compared to the excitation light rays EB and EV. Furthermore, it is understood that the transmitted light R from the second light source 50 has a wavelength range different from the fluorescences λ1B and λ1V. Since the transmitted light R and the fluorescences λ1B and λ1V have different wavelength ranges, two fluorescences and one transmitted light can be imaged with the imaging element 26, as will be described below.

The fluorescences λ1B and λ1V and the transmitted light R are transmitted through the objective lens 16 and the dichroic mirror 20 having the spectroscopic characteristics shown in FIG. 16, and reach the second multi-bandpass filter 22. Here, as shown in FIG. 16, according to the spectroscopic characteristics of the dichroic mirror 20, the transmittances of the wavelength ranges of the fluorescences λ1B and λ1V and the transmitted light R are high. Therefore, the fluorescences λ1B and λ1V and the transmitted light R are transmitted through the dichroic mirror 20. FIG. 18 is a graph showing a spectroscopic spectrum of the second multi-bandpass filter 22, and in the graph, the axis of ordinate represents the transmittance (%) while the axis of abscissa represents the wavelength (nm). As shown in FIG. 18, the second multi-bandpass filter 22 has spectroscopic spectra of F2B, F2V, and FR, which transmits only wavelength ranges that are narrower than the wavelength ranges of the fluorescences λ1B and λ1V and the transmitted light R, and the second multi-bandpass filter 22 can be configured as a triple bandpass filter.

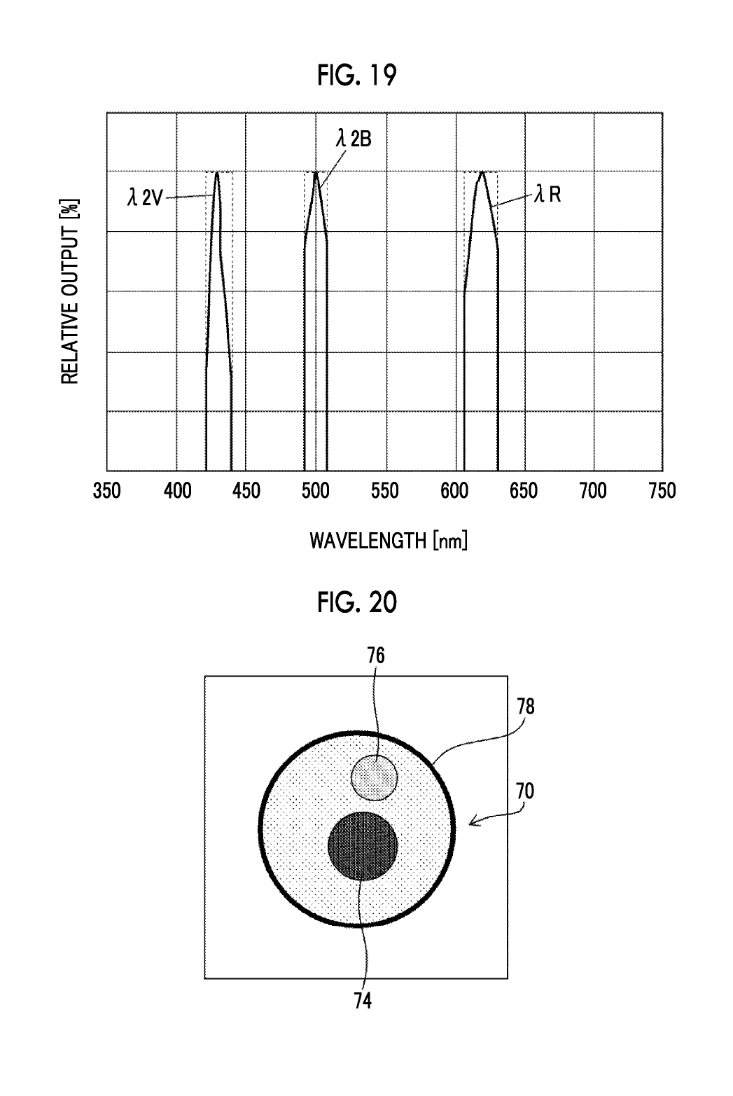

The fluorescence λ1B that has been emitted from the cell C and transmitted through the dichroic mirror 20 is treated such that light rays in unnecessary wavelength ranges are cut off upon being transmitted through the second multi-bandpass filter 22. The fluorescence λ1V that has been emitted from the cell C and transmitted through the dichroic mirror 20 is treated such that light rays in unnecessary wavelength ranges are cut off upon being transmitted through the second multi-bandpass filter 22. Transmitted light R that has been transmitted through the cell C and the dichroic mirror 20 is treated such that light rays in unnecessary wavelength ranges are cut off upon being transmitted through the second multi-bandpass filter 22. FIG. 19 is a graph of spectra of the fluorescences and the transmitted light that have been transmitted through the second multi-bandpass filter 22, and in the graph, the axis of ordinate represents the relative output (%) while the axis of abscissa represents the wavelength (nm). The fluorescence λ1B has its unnecessary wavelength ranges cut off by the second multi-bandpass filter 22, and the fluorescence λ2B having a narrower wavelength range than the fluorescence λ1B is outputted from the second multi-bandpass filter 22. The fluorescence λ1V has its unnecessary wavelength ranges cut off by the second multi-bandpass filter 22, and the fluorescence λ2V having a narrower wavelength range than the fluorescence λ1V is outputted from the second multi-bandpass filter 22. Transmitted light R has its unnecessary wavelength ranges cut off by the second multi-bandpass filter 22, and transmitted light λR having a narrower wavelength range than transmitted light R is outputted from the second multi-bandpass filter 22.

In the step of imaging, the fluorescences λ2B and λ2V and the transmitted light λR, which have been transmitted through the second multi-bandpass filter 22, are imaged by an imaging element 26 having a plurality of sub-pixels in each pixel. Regarding the imaging element 26, a single plate type color imaging element having a red filter, a green filter, and a blue filter similarly to the first embodiment can be used.

The intensities of the fluorescences and the intensity of the transmitted light imaged by the imaging element 26 are inputted and stored in, for example, a control unit 28. The intensities of the fluorescences and the intensity of the transmitted light are computed by the control unit 28, and thus a color image is obtained.

FIG. 20 is a conceptual diagram of an image of a fluorescence emitted from the cell C and a phase difference image, the image being obtained by the control unit 28 based on the intensities of fluorescences obtained by the imaging element 26. FIG. 20 displays a nucleated red blood cell 70. In regard to the nucleated red blood cell 70, there are occasions in which the fluorescence of an immature red blood cell does not fluoresce uniformly over the entire red blood cell but fluoresces locally. In this case, the shape of the nucleated red blood cell 70 cannot be recognized through the fluorescence. In such a case, the shape can be recognized by taking a bright field image and superimposing the bright field image with the fluorescence. A bright field image is preferable because clearer recognition of the shape is enabled by further obtaining a phase difference image.

In FIG. 20, the nucleus 74 in the nucleated red blood cell 70 emits, for example, blue light due to a fluorescent dye, and HbF76 (fetal hemoglobin) in the nucleated red blood cell 70 emits green light due to a fluorescent dye. For the external contour shape 78 of the nucleated red blood cell 70, a phase difference image is imaged by utilizing transmitted light.

In the present embodiment, since fluorescences of different wavelengths emitted from the cell C and transmitted light from the second light source as described above can be simultaneously imaged by one-time imaging, the time for imaging the cell C can be shortened.

### EXPLANATION OF REFERENCES

10: imaging device
12: First light source
14: table
16: objective lens
18: first multi-bandpass filter
20: dichroic mirror
22: second multi-bandpass filter
24: filter group
26: imaging element
28: control unit
40: container
42: housing unit
44: opening
46: lateral surface
48: holding surface
50: second light source
60: white blood cell
62, 72, 82: surface
64, 74: nucleus
70: nucleated red blood cell
76: HbF
78: external contour shape
80: red blood cell
260: pixel
261, 262, 263, 264: sub-pixel
EB, EG, EV: excitation light
R, λR: transmitted light
λ1B, λ1G, λ1V, λ2B, λ2G, λ2V: fluorescence

What is claimed is:

1. An imaging device comprising:
at least one housing unit for holding and housing a cell that has been stained with a plurality of kinds of fluorescent dyes emitting fluorescences of different wavelengths on a flat holding surface;
a first light source simultaneously emitting light rays having a plurality of wavelengths;
a filter group including a first multi-bandpass filter, a dichroic mirror, and a second multi-bandpass filter,
the first multi-bandpass filter selectively transmitting excitation light rays having a plurality of wavelengths that excite the plurality of kinds of fluorescent dyes from among the light rays having the plurality of wavelengths emitted from the first light source,
the dichroic mirror radiating the excitation light rays having the plurality of wavelengths that have been transmitted through the first multi-bandpass filter toward the cell and transmitting the fluorescences of different wavelengths that are emitted from the cell due to the excitation light rays having the plurality of wavelengths, and
the second multi-bandpass filter transmitting the fluorescences of different wavelengths that have been transmitted through the dichroic mirror;
an objective lens condensing the excitation light rays having the plurality of wavelengths and amplifying the fluorescences of different wavelengths; and
an imaging element having a plurality of sub-pixels in each pixel, and imaging the fluorescences of different wavelengths that have been transmitted through the second multi-bandpass filter.

2. The imaging device according to claim 1, wherein the imaging element is a color imaging element.

3. The imaging device according to claim 2, wherein the color imaging element is a single plate type imaging element having a red filter, a green filter, and a blue filter.

4. The imaging device according to claim 1, wherein the first light source, the filter group, and the imaging element are disposed on the opposite side of the holding surface with respect to the housing unit.

5. The imaging device according to claim 2, wherein the first light source, the filter group, and the imaging element are disposed on the opposite side of the holding surface with respect to the housing unit.

6. The imaging device according to claim 3, wherein the first light source, the filter group, and the imaging element are disposed on the opposite side of the holding surface with respect to the housing unit.

7. The imaging device according to claim 1, wherein the first multi-bandpass filter and the second multi-bandpass filter are each formed from a triple bandpass filter.

8. The imaging device according to claim 2, wherein the first multi-bandpass filter and the second multi-bandpass filter are each formed from a triple bandpass filter.

9. The imaging device according to claim 3, wherein the first multi-bandpass filter and the second multi-bandpass filter are each formed from a triple bandpass filter.

10. The imaging device according to claim 4, wherein the first multi-bandpass filter and the second multi-bandpass filter are each formed from a triple bandpass filter.

11. The imaging device according to claim 1, wherein the first light source is composed of a plurality of light sources.

12. The imaging device according to claim 11, further comprising a control unit for controlling the amounts of light from the plurality of light sources independently.

13. The imaging device according to claim 11, wherein the first light source is a light source including at least a green light emitting diode, a blue light emitting diode, or a violet light emitting diode.

14. The imaging device according to claim 1, wherein the housing unit is one of the housing units of a container having a plurality of housing units.

15. An imaging device comprising:
at least one housing unit for holding and housing a cell that has been stained with a plurality of kinds of fluorescent dyes emitting fluorescences of different wavelengths on a flat holding surface;
a first light source simultaneously emitting light rays having a plurality of wavelengths;
a second light source disposed on the opposite side of the first light source with respect to the housing unit and emitting transmitted light having a single wavelength;
a filter group including a first multi-bandpass filter, a dichroic mirror, and a second multi-bandpass filter,
the first multi-bandpass filter selectively transmitting excitation light rays having a plurality of wavelengths that excite the plurality of kinds of fluorescent dyes from among the light rays having the plurality of wavelengths emitted from the first light source,
the dichroic mirror radiating the excitation light rays having the plurality of wavelengths that have been transmitted through the first multi-bandpass filter toward the cell and transmitting the fluorescences of different wavelengths that are emitted from the cell due to the excitation light rays having the plurality of wavelengths while also transmitting the transmitted light having the single wavelength emitted from the second light source, and
the second multi-bandpass filter transmitting the fluorescences of different wavelengths that have been transmitted through the dichroic mirror and the transmitted light having the single wavelength;
an objective lens condensing the excitation light rays having the plurality of wavelengths and amplifying the fluorescences of different wavelengths and the transmitted light having the single wavelength; and
an imaging element having a plurality of sub-pixels in each pixel, and imaging the fluorescences of different wavelengths that have been transmitted through the second multi-bandpass filter and the transmitted light having the single wavelength.

16. The imaging device according to claim 15, wherein the imaging element is a color imaging element.

17. The imaging device according to claim 16, wherein the color imaging element is a single plate type imaging element having a red filter, a green filter, and a blue filter.

18. The imaging device according to claim 15, wherein the first light source is a light source including two light emitting diodes selected from a group consisting of a green light emitting diode, a blue light emitting diode, and a violet light emitting diode.

19. An imaging method using the imaging device according to claim 1 comprising:
a step of housing the cell that has been stained with the plurality of kinds of fluorescent dyes emitting fluorescences of different wavelengths in at least one housing unit having the holding surface for holding the cell on the flat surface;
a step of simultaneously emitting light rays having the plurality of wavelengths from the first light source;
a step of selectively transmitting excitation light rays having the plurality of wavelengths that excite the plurality of kinds of fluorescent dyes from among the light rays having the plurality of wavelengths emitted from the first light source, through the first multi-bandpass filter, radiating the excitation light rays having the plurality of wavelengths that have been transmitted through the first multi-bandpass filter, toward the cell by the dichroic mirror, transmitting the fluorescences of different wavelengths emitted from the cell due to the excitation light rays having the plurality of wavelengths through the dichroic mirror, and transmitting the fluorescences of different wavelengths that have been transmitted through the dichroic mirror, through the second multi-bandpass filter; and
a step of imaging the fluorescences of different wavelengths that have been transmitted through the second multi-bandpass filter, using an imaging element having the plurality of sub-pixels in each pixel.

20. An imaging method using the imaging device according to claim 15 comprising:
a step of housing the cell that has been stained with the plurality of kinds of fluorescent dyes emitting fluorescences of different wavelengths in at least one housing unit having the holding surface for holding the cell on the flat surface;
a step of simultaneously emitting light rays having the plurality of wavelengths from the first light source, and emitting transmitted light having the single wavelength from the second light source that is disposed on the opposite side of the first light source with respect to the housing unit;
a step of selectively transmitting excitation light rays having the plurality of wavelengths that excite the plurality of kinds of fluorescent dyes from among the light rays having the plurality of wavelengths emitted from the first light source, through the first multi-bandpass filter, radiating the excitation light rays having the plurality of wavelengths that have been transmitted through the first multi-bandpass filter toward the cell by the dichroic mirror, transmitting the fluorescences of different wavelengths that have been emitted from the cell due to the excitation light rays having the plurality of wavelengths and the transmitted light having the single wavelength that is emitted from the second light source through the dichroic mirror, and transmitting the fluorescences of different wavelengths that have been transmitted through the dichroic mirror and the transmitted light having the single wavelength emitted from the second light source, through a second multi-bandpass filter; and
a step of imaging the fluorescences of different wavelengths that have been transmitted through the second multi-bandpass filter and the transmitted light having the single wavelength emitted from the second light source, using an imaging element having the plurality of sub-pixels in each pixel.
